(19) United States
(12) Patent Application Publication
(10) Pub. No.: US 2023/0285670 A1
(43) Pub. Date: Sep. 14, 2023

(54) ADAPTIVE BASAL PORTION VALUE IN AN AUTOMATED MEDICAMENT DELIVERY DEVICE BASED ON HISTORIC MEDICAMENT DELIVERY FOR A USER

(71) Applicant: INSULET CORPORATION, Acton, MA (US)

(72) Inventors: Joon Bok LEE, Acton, MA (US); Ashutosh ZADE, San Diego, CA (US); Yibin ZHENG, Hartland, WI (US); Jason O'CONNOR, Acton, MA (US); Mengdi LI, Westford, MA (US)

(21) Appl. No.: 18/119,059

(22) Filed: Mar. 8, 2023

Related U.S. Application Data
(60) Provisional application No. 63/369,790, filed on Jul. 29, 2022.
(60) Provisional application No. 63/318,217, filed on Mar. 9, 2022.

(51) Int. Cl. A61M 5/172; A61M 5/142

(52) U.S. Cl. CPC A61M 5/1723; A61M 5/14244; A61M 2202/0486; A61M 2205/50; A61M 2205/52

(57) ABSTRACT

The exemplary embodiments may provide adaptivity in the basal portion value. The exemplary embodiments may also provide adaptivity of the total daily medicament (TDM) delivery amount. The adaptivity provided by the exemplary embodiments helps to customize medicament delivery to the needs of the user of a medicament delivery device. The exemplary embodiments may adapt the basal ratio for the user based on historical medicament delivery data for the user. In addition, the exemplary embodiments may update the basal ratio based upon more recent trends that diverge from the historical medicament delivery data. The degree of adaptivity provided by the exemplary embodiments may be bound to not exceed an upper threshold and/or a lower threshold. The degree of adaptivity may be modulated by controlling the rate of adaptivity too small increments each time the basal ratio is updated.

### RELATED APPLICATIONS

This application claims the benefit of U.S. Provisional Patent Application No. 63/318,217, filed Mar. 9, 2022 and U.S. Provisional Patent Application No. 63/369,790, filed Jul. 29, 2022, the contents of which are incorporated herein by reference in their entirety.

### BACKGROUND

Automated insulin delivery (AID) devices, like insulin pumps, enable delivery of insulin boluses as well as basal insulin deliveries. The basal insulin deliveries provide small amounts of insulin at periodic intervals on an ongoing basis. The insulin boluses are provided to compensate for blood glucose increases that may result from ingesting meals or to compensate for blood glucose levels being too high.

Conventionally, AID devices determine how much basal insulin to deliver to a user based upon total daily insulin (TDI). TDI is the sum of all insulin deliveries to the user on a daily basis. TDI may be initially set for a user based upon the user's weight. The quantity of insulin delivered by basal deliveries per day is derived from TDI. Traditionally, the quantity of insulin of delivered by basal deliveries per day is set at half of TDI for the user.

### SUMMARY

In accordance with an inventive aspect, a medicament delivery system includes a non-transitory storage for storing computer programming instructions and a processor for executing the computer programming instructions. The computer programming instructions cause the processor to calculate a new basal ratio for a user, where the new basal ratio specifies what portion of total daily medicament for the user is to be delivered by basal medicament deliveries based on a previous basal ratio for the user and an adjustment. The adjustment includes a ratio of amounts of a medicament delivered as basal medicament deliveries to the user via an automated medicament delivery device over a period to a total amount of medicament delivered to the user over the period, and at least one adjustment restriction that restricts how much the new basal ratio may vary from the previous basal ratio. The computer programming instructions also cause the processor to determine a dosage for a next basal medicament delivery by the automated medicament delivery device to the user using the new basal ratio.

The medicament may be insulin. The medicament may include at least one of a glucagon like peptide-1 (GLP-1) agonist, pramlintide or another agent that affects a glucose level of the user. The determining of the dosage for the next basal medicament delivery may include multiplying a total daily insulin for the user by the new basal ratio. At least one adjustment restriction may include a maximum and/or minimum value that the adjustment may assume. At least one adjustment restriction may limit a difference between the previous basal ratio and the new basal ratio to a predetermined fixed amount. At least one adjustment restriction may be a scaling factor that scales the adjustment.

In accordance with another inventive aspect, a medicament delivery system includes a non-transitory storage for storing computer programming instructions and a processor for executing the computer programming instructions. The computer programming instructions cause the processor to determine an average amount of insulin delivered to a user per day to compensate for carbohydrates ingested in meals and to determine an average amount of insulin delivered to the user per day to correct for high glucose experienced by the user. The computer programming instructions also cause the processor to determine an average amount of insulin delivered by the user as basal deliveries and to compute the sum of the average amount of insulin delivered to a user per day to compensate for carbohydrates ingested in meals, the average amount of insulin delivered to the user per day to correct for high glucose experienced by the user, and the average amount of insulin delivered by the user as basal deliveries. In addition, the computer programming instructions cause the processor to determine a basal portion factor as a ratio of the average amount of insulin delivered by the user as basal deliveries to the sum and to determine an updated basal delivery rate for the user using the basal portion factor.

The determining of the updated basal delivery rate for the user may include determining an updated hourly basal delivery rate for the user. Alternative interval rates can be determined, such as every 2 hours, every 30 minutes, every 15 minutes, or every 5 minutes, for example. The determining of the hourly basal delivery rate may include determining a product of total daily insulin (TDI) for the user with the basal portion factor and dividing the product by 24 to get the hourly basal delivery rate. Other intervals for the basal delivery rate may be calculated in a similar manner. The method may also include updating the basal portion factor and updating the basal delivery rate for the user using the updated basal portion factor. The updating of the basal portion factor may comprise increasing the basal portion factor by a first predetermined fixed amount if the basal portion factor is below a threshold. The updating of the basal portion factor may include decreasing a basal portion factor by a second predetermined fixed amount if the basal portion factor is above a threshold.

In accordance with an additional inventive aspect, a medicament delivery system includes a display and a non-transitory storage for storing computer programming instructions. The medicament delivery system also includes a processor for executing the computer programming instructions. The computer programming instructions cause the processor to compare an average meal bolus portion of total daily insulin for a patient over most recent days with a historical average meal bolus portion of total daily insulin for the patient. If the average meal bolus portion of total daily insulin over most recent days is higher than the historical average meal bolus portion of total daily insulin by a first percentage, the processor generates a suggestion on the display to the user to lower an amount of insulin for meal boluses by the first percentage. If the average meal bolus portion of total daily insulin over most recent days is lower than the historical average meal bolus portion of total daily insulin by a second percentage, the processor generates a suggestion on the display to the user to increase an amount of insulin for meal boluses by the first percentage.

The medicament delivery system may include an insulin pump, a controller for an insulin pump or a portable computing device, such as a smartphone. The computer programming instructions may further cause the processor to calculate a historical average basal daily insulin delivery to the patient. The computer programming instructions may further cause the processor to calculate a historical average meal bolus daily insulin delivery to the patient. The computer programming instructions further may cause the processor to calculate historical average total daily insulin for the patient as the sum of the historical average basal daily insulin delivery to the patient and the historical average meal bolus daily insulin delivery to the patient. The computer programming instructions may further cause the processor to calculate the historical average meal bolus portion of total daily insulin for the patient as a ratio of historical average meal bolus daily insulin delivery to the patient to the historical average total daily insulin for the patient. The first percentage may equal the second percentage.

In accordance with a further inventive facet, a medicament delivery device for delivering a medicament to a user includes a non-transitory storage medium for storing computer programming instructions. The medicament delivery device additionally includes a processor configured for executing the computer programming instructions to cause the processor to use a first basal portion value that is indicative of a portion of a total daily medicament (TDM) of a user to determine an amount of basal medicament to be delivered by the medicament delivery device during a first interval of operation. The instructions cause the processor to determine an adapted basal portion value for a next interval of operation after the first interval of operation based on a magnitude of recent basal deliveries by the medicament delivery device and cause the processor to use the adapted basal portion value to determine amounts of basal medicament to be delivered by the medicament delivery device during the next interval of operation. The instructions cause the processor also to cause the determined amount of basal medicament deliveries to be delivered by the medicament delivery device during the next interval of operation.

The determining of the adapted basal portion value for the next interval of operation may comprise assigning a first weight to the first basal portion value for the first interval of operation to obtain a first weighted value, determining a calculated ratio of a total amount of basal medicament delivered to the user by the medicament delivery device during a period of operation to a total amount of basal medicament that would be delivered over the period if an initial basal portion value is used, applying a second weight to the calculated ratio to produce a second weighted value, and summing the first weighted value and the second weighted value to determine the adapted basal portion value. The initial basal portion value may be a basal/TDM ratio, and the basal/TDM ratio for the initial basal portion value may be 0.5. The initial basal portion value may be tailored to an insulin sensitivity of the user. The first weight may be at least four times the second weight. The medicament may be insulin, a glucagon-like peptide 1 (GLP-1) agonist, pramlintide or a coformulation of at least two of the foregoing. The medicament delivery device may be an insulin patch pump. The determining of an adapted basal portion value for a next interval of operation after the first interval of operation may include calculating TDM based on recent medicament deliveries and using the calculated TDM in determining the adapted basal portion value.

In accordance with yet another inventive facet, a medicament delivery device for delivering a medicament to a user may include a non-transitory storage medium for storing computer programming instructions and a processor configured for executing the computer programming instructions to cause the processor to perform the following: use a first basal portion value indicative of a portion of a total daily medicament (TDM) of a user that is delivered in basal form to determine an amount of basal medicament to be delivered by the medicament delivery device during a first interval of operation, determine an adapted basal portion value for a next interval of operation after the first interval of operation based on a magnitude of recent bolus deliveries by the medicament delivery device, use the adapted basal portion value to determine an amount of basal medicament to be delivered by the medicament delivery device during the next interval of operation, and cause the determined amount of basal medicament to be delivered by the medicament delivery device during the next interval of operation.

The first basal portion value may be a basal to TDM ratio with a value of 0.5. The determining of the adapted basal portion value for the next interval of operation may include assigning a first weight to the first basal portion value for the first interval of operation to obtain a first weighted value, determining a ratio of a total amount of bolus medicament delivered to the user by the medicament delivery device during a period of operation to a total amount of bolus medicament that would be delivered over the period if an expected basal portion value is used, subtracting the determined ratio from one to yield a difference, applying a second weight to the difference to produce a second weighted value, and summing the first weighted value and the second weighted value to determine the adapted basal portion value.

The first basal portion value may be tailored to a medicament sensitivity of the user. The medicament may be insulin, a glucagon-like peptide-1 (GLP-1) agonist, pramlintide or a coformulation of two or more of the foregoing. The medicament delivery device may be an insulin patch pump. The first weight may be at least four times the second weight.

In accordance with yet another inventive aspect, a method of managing basal insulin delivery dosage amounts performed by a processor of an insulin delivery device is performed. The method includes storing basal insulin delivery dosage amounts delivered by the insulin delivery device to a user in a non-transitory computer-readable storage medium. The method also includes, with the processor, determining the aggregate basal insulin delivery amount for a time horizon based on at least some of the stored basal insulin delivery dosage amounts and, with the processor, determining a ratio of the determined aggregate basal delivery amount to an expected aggregate basal delivery amount for the time horizon. The method further includes weighting the ratio and weighting a previous ratio of basal to TDM, or in the case of used hereafter, total daily insulin (TDI) that was used by the processor to determine basal delivery dosage amounts for a previous time interval. The method additionally includes, with the processor, summing the weighted ratio and the weighted previous ratio to produce a new ratio of basal insulin deliveries to TDI for a next interval of time and, with the processor, using the new ratio of basal insulin deliveries to TDI to determine basal insulin delivery amounts for the next interval of time. The method further includes causing the insulin delivery device to deliver the determined basal insulin delivery amounts over the next interval of time.

The insulin delivery device may have cycles of a fixed length, and the expected aggregate basal delivery amount for the time horizon may be one half of the TDI divided by the ratio of a number of cycles in a day to the number of cycles in the time horizon. The method may further include calculating TDI from insulin deliveries to the user by the insulin delivery device over the time horizon. Weighing the previous ratio of basal to TDI may include assigning the previous ratio a weight that is at least four times as large as a weight assigned to the ratio. The previous basal to TDI ratio may have been customized to the user.

### BRIEF DESCRIPTION OF THE DRAWINGS

FIG. 1 depicts a medicament delivery system suitable for exemplary embodiments.

FIG. 2 depicts a flowchart of illustrative steps that may be performed in exemplary embodiments to adapt a basal portion value to a user from a medicament delivery device.

FIG. 3 depicts a flowchart of illustrative steps that may be performed in exemplary embodiments to determine a new basal portion value according to a first option.

FIG. 4 depicts a flowchart of illustrative steps that may be performed in exemplary embodiments to determine a basal dosage for delivery by the medicament delivery device.

FIG. 5 depicts a flowchart of illustrative steps that may be performed in exemplary embodiments to determine a new adjustment factor having a minimum and a maximum.

FIG. 6A depicts a flowchart of illustrative steps that may be performed in exemplary embodiments to determine a new adjustment factor by averaging an experienced basal portion value with an ideal basal portion value.

FIG. 6B depicts a flowchart of illustrative steps that may be performed in exemplary embodiments to determine a new basal portion value according to a second option.

FIG. 7 depicts a flowchart of illustrative steps that may be performed in exemplary embodiments to determine an adjustment factor with fixed value options.

FIG. 8 depicts a flowchart of illustrative steps that may be performed in exemplary embodiments to determine an adjustment factor with scaled values.

FIG. 9 depicts a flowchart of illustrative steps that may be performed in exemplary embodiments to generate a user notification of the need for an adjustment in bolus dosages.

FIG. 10 depicts a flowchart of illustrative steps that may be performed in exemplary embodiments to determine an hourly basal rate.

FIG. 11 depicts a flowchart of illustrative steps that may be performed in exemplary embodiments to make incremental adjustments to a basal portion value.

FIG. 12 depicts a flowchart of illustrative steps that may be performed in exemplary embodiments to suggest adjustments in bolus amounts based on an average basal portion value.

FIG. 13 depicts a flowchart 1300 of illustrative steps that may be performed in exemplary embodiments to adapt the basal portion value and TDM for a user.

FIG. 14 depicts a flowchart of illustrative steps that may be performed in exemplary embodiments to adjust the basal portion value for the shortened time intervals.

FIG. 15A depicts a flowchart of illustrative steps that may be performed in exemplary embodiments to determine the adapted basal portion value from recent medicament delivery history of the user.

FIG. 15B depicts a flowchart of illustrative steps that may be performed in exemplary embodiments to adapt the basal portion value for a user.

FIG. 16 depicts a flowchart of steps that may be performed in exemplary embodiments to adapt TDM for a user.

FIG. 17 depicts a flowchart of illustrative steps that may be performed in exemplary embodiments to use the adapted TDM in determining the basal portion value.

### DETAILED DESCRIPTION

The exemplary embodiments may provide adaptivity in the basal portion value (e.g., a “basal ratio” of basal deliveries per day to total daily medicament (TDM) amount) that identifies what portion of the total deliveries of a medicament per day to a user are basal medicament deliveries. More generally, the basal portion value is indicative of the total dosage of basal medicament deliveries over a time period, such as a day. “Basal ratio” may be used herein as an example of a basal portion value. That said, it should be appreciated that the basal portion value need not be expressed solely as a basal ratio. For example, the basal portion value may be a decimal value or may be an absolute quantity of basal medicaments per day or other time period in some embodiments.

The exemplary embodiments may also provide adaptivity of the TDM to adjust to changing insulin needs of the user. As was mentioned above, conventional medicament delivery devices, such as AID devices, may operate with a fixed TDI and a fixed basal portion value. A difficulty with operating with a fixed TDI and a fixed basal portion value is that the TDI and/or fixed basal portion value may not be well-suited for the user. The user, for example, may be better served with a higher basal portion value or a lower basal portion value. Similarly, the TDI may be either too high or too low for the user.

The exemplary embodiments may adapt the basal portion value for the user based on historical medicament delivery data for the user. In addition, the exemplary embodiments may update the basal portion value based upon more recent trends that diverge from the historical medicament delivery data. The degree of adaptivity provided by the exemplary embodiments may be bound to not exceed an upper threshold and/or a lower threshold. Further, the degree of adaptivity may be modulated by controlling the rate of adaptivity too small increments each time the basal ratio is updated. The increments may be fixed in size, adjusted by a scaling factor or the like.

In some exemplary embodiments, the dosage size of a medicament bolus delivered to the user may be adjusted or suggestions may be generated to adjust the bolus based on recent medicament bolus history and historical medicament delivery data. The dosage of the medicament bolus may be adjusted upwards or downwards based on how much the ratio of average daily medicament boluses to TDM ratio has changed from a historical average of the value for the ratio. Thus, the bolus dosages may be adjusted to reflect a change in the split of average daily basal medicament deliveries versus average daily bolus medicament deliveries.

Exemplary embodiments may adjust the basal portion value of the user based upon recent medicament sensitivity of the user. The adjustments may be made on an on-going basis over the life of the medicament delivery device. Moreover, the adjustments may be made quickly to be more responsive to changing medicament needs of the user. For instance, some insulin pumps have an effective lifetime of about three days. Instead of fixing a basal portion value and/or TDM for a user solely when the insulin pump is first attached to the user and activated, as with some conventional medicament delivery devices, exemplary embodiments may adjust the basal portion value and/or TDM at shorter time intervals, such as once every hour, once every three hours, once every twelve hours or the like. Where the medicament is insulin, for example, this enables the medicament delivery device to better account for changes in insulin sensitivity of the user, such as due to exercising or due to the dawn phenomenon, which may produce an abnormal change in insulin sensitivity between 2 AM to 8 AM in some users.

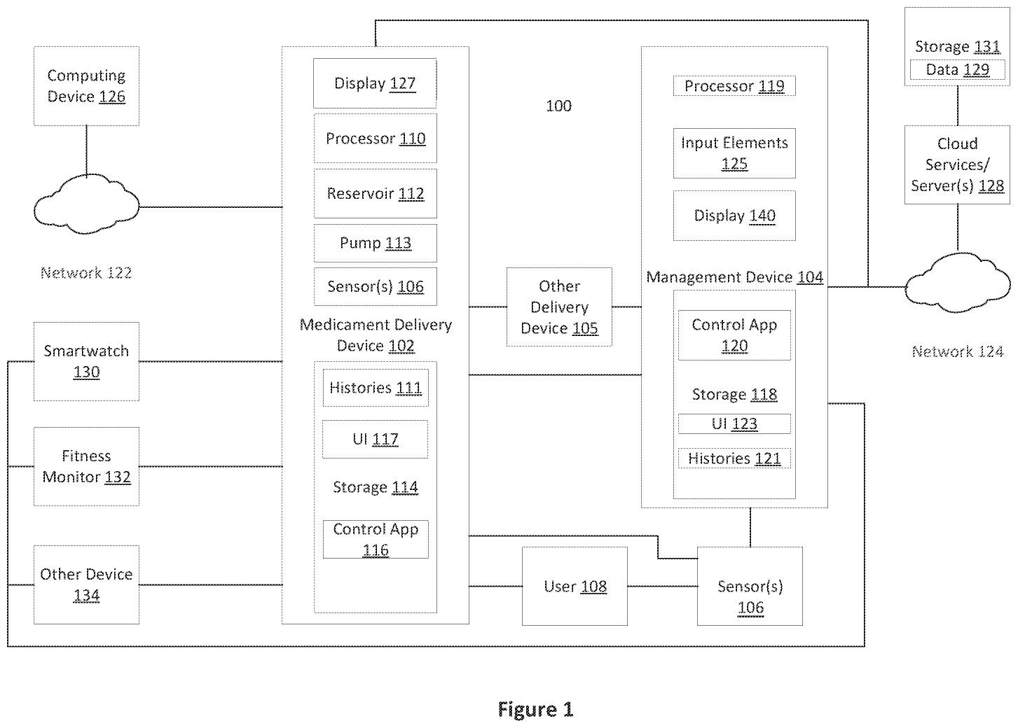

FIG. 1 depicts an illustrative medicament delivery system 100 that is suitable for delivering a medicament to a user 108 in accordance with the exemplary embodiments. The medicament delivery system 100 includes a medicament delivery device 102. The medicament delivery device 102 may be a wearable device that is worn on the body of the user 108 or carried by the user. The medicament delivery device 102 may be directly coupled to a user (e.g., directly attached to a body part and/or skin of the user 108 via an adhesive or the like) with no tubes and an infusion location directly under the medicament delivery device 102, or carried by the user (e.g., on a belt or in a pocket) with the medicament delivery device 102 connected to an infusion site where the medicament is injected using a needle and/or cannula. In a preferred embodiment, a surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of a medicament to the user 108 per a control approach like that described herein. The control application may provide the adaptability in the basal ratio, the total daily medicament amount and boluses described below. The storage 114 may hold histories 111 for a user, such as a history of basal deliveries, a history of bolus deliveries, and/or other histories, such as a meal event history, exercise event history, glucose level history and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include one or more housings for housing its various components including a pump 113, a power source (not shown), and a reservoir 112 for storing a medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site.

There may be one or more communications links with one or more devices physically separated from the medicament delivery device 102 including, for example, a management device 104 of the user and/or a caregiver of the user, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN) or a combination therein. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions and/or physical attributes of the user 108. The sensor(s) 106 may be physically separate from the medicament delivery device 102 or may be an integrated component thereof. The sensor(s) 106 may include, for example, glucose monitors, such as continuous glucose monitors (CGM's) or non-invasive glucose monitors. The sensor(s) 106 may include ketone sensors, analyte sensors, heart rate monitors, breathing rate monitors, motion sensors, temperature sensors, perspiration sensors, blood pressure sensors, alcohol sensors or the like.

The medicament delivery system 100 may or may not also include a management device 104. In some embodiments, no management device is needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensor(s) 106. The management device 104 may be any portable electronic device including, for example, a dedicated device, a smartphone, a smartwatch or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. The control application 120 may be responsible for controlling the medicament delivery device 102, such as by controlling the AID delivery of insulin to the user 108. In some exemplary embodiments, the control application 120 provides the adaptability described herein. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when it is a touchscreen. The management device 104 may further include input elements 125, such as a keyboard, button, knobs, or the like, for receiving input form the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user, such as to request a change or pause in dosage or to request, initiate, or confirm delivery of a bolus of a medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data. Another delivery device 105, such as a medicament delivery pen, ma be provided for also delivering medicament to the user 108.

A wide variety of medicaments may be delivered by the medicament delivery device 102 and delivery device 105. The medicament may be insulin for treating diabetes. The medicament may be glucagon for raising a user's glucose level. The medicament may also be a glucagon-like peptide (GLP)-1 receptor agonists for lowering glucose or slowing gastric emptying, thereby delaying spikes in glucose after a meal. Alternatively, the medicament delivered by the medicament delivery device 102 may be one of a pain relief agent, a chemotherapy agent, an antibiotic, a blood thinning agent, a hormone, a blood pressure lowering agent, an antidepressant, an antipsychotic, a statin, an anticoagulant, an anticonvulsant, an antihistamine, an anti-inflammatory, a steroid, an immunosuppressive agent, an antianxiety agent, an antiviral agents, a nutritional supplement or a vitamin. The medicament may be a coformulation of some of those medicaments listed above.

The functionality described below for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 134.

In the closed loop mode, the control application 116, 120 determines the medicant delivery amount for the user 108 on an ongoing basis based on a feedback loop. For an insulin delivery device, the aim of the closed loop mode is to have the user's glucose level at a target glucose level.

As was mentioned above, the exemplary embodiments may provide adjustability to the TDM, the basal portion value and the bolus medicament delivery dosages provided by the medicament delivery device 102. The control applications 116 and 120 may be configured to provide such adaptability as will be described below. There are a number of ways of providing the adaptability in exemplary embodiments as described below.

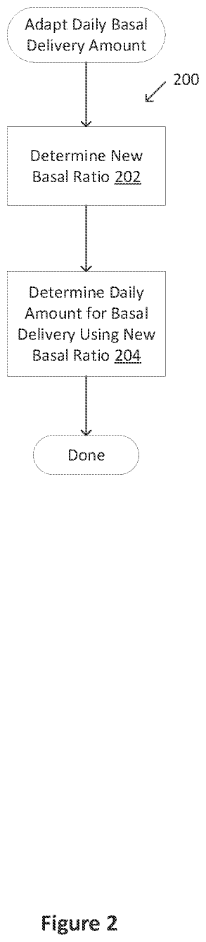

FIG. 2 depicts a flowchart 200 of illustrative steps that may be performed to adjust the daily basal delivery amounts of medicament in exemplary embodiments. At 202, the new basal portion value expressed as a basal ratio may be determined based upon medicament delivery data gathered over a recent period of days. At 204, once the new basal ratio has been determined, a new daily basal delivery amount may be determined using the new basal ratio as will be detailed below. The basic equation for this determination is:

DB_{new}=BR_{new}*TDM_{new} (Eq. 1)

where DB_{new} is the new daily basal delivery amount, BR_{new} is the new basal ratio, and TDM_{new} is the new total daily medicament amount.

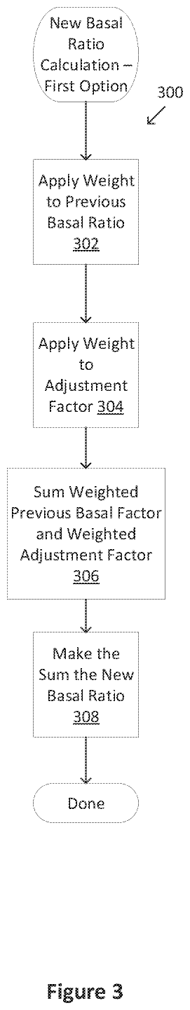

FIG. 3 depicts a flowchart 300 of illustrative steps that may be performed in a first option to determine a new basal portion value expressed as a basal ratio in exemplary embodiments. A suitable equation for calculating the new basal ratio, BR_{new} is:

BR_{new}=(1−Aᵣ*N_{days})*BR_{old}+Aᵣ*N_{days}*Adjustment Factor (Eq. 2)

where N_{days} is the number of days in the time period upon which the adjustment is made and BR_{old} is the previous basal ratio that is being adjusted. The adjustment factor is based on the recently gathered data regarding deliveries of medicament and seeks to adjust the basal ratio toward the experienced basal ratio that is reflected in the gathered data. At 302, a weight is applied to the previous basal ratio BR_{old}. In the above Equation 2, the weight is (1−Aᵣ*N_{days}), with the adaptivity rate Aᵣ controlling the rate at which the adjustment factor is incorporated into the new basal rate, with a nominal value of 0.2. The adaptivity rate may be variable. The influence of the previous basal ratio is higher when the period is shorter. For example, if the period contained only a single day, the weight would be 0.8 (i.e., 1−0.2*1), whereas is if the period is 3 days, the weight decreases to 0.4. At 304, the weight is applied to the adjustment factor. The adjustment factor, which reflects the newer gathered medicament delivery data, has an inversely configured weight relative to BR_{old} of Aᵣ*N_{days}, with the same nominal value for adaptivity rate of 0.2. The weight of the adjustment factor increases as the number of days in the time period increase. For a single day, the weight is 0.2, but the weight increases to 0.6 when the number of days in the period is 3 days. At 306, the weighted previous basal ratio (e.g., (1−Aᵣ*N_{days})*BR_{old}) is added to the weighted adjustment factor (e.g., Aᵣ*N_{days}*Adjustment Factor). At 308, the sum is made the new basal ratio BR_{new}.

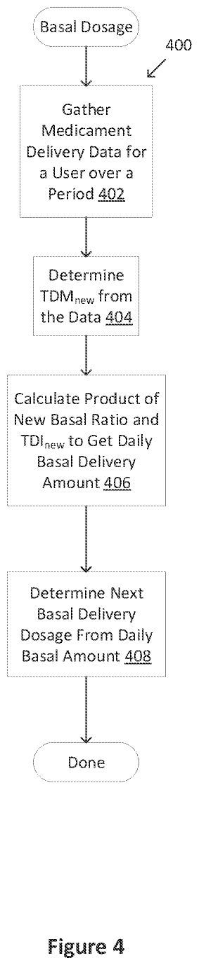

FIG. 4 depicts a flowchart 400 of illustrative steps that may be performed in exemplary embodiments to determine the dosage for basal delivery using the new basal ratio (see 204 in FIG. 2). At 402, the medicament delivery data for a user is gathered over a recent period. This recent period is a period after the extended period over which the historical data has been previously gathered. At 404, the new total daily medicament (TDM_{new}) is determined from the gathered data for the recent period. A suitable equation for TDM_{new} is:

TDM_{new}=1/nΣₖ₌₀ⁿMedicament(basal)+Medicament(bolus) (Eq. 3)
where n=number of days of recent period

In order to calculate the TDM_{new}, the sum of bolus medicament deliveries and basal medicament deliveries (see Σₖ₌₀ⁿ Medicament(basal)+Medicament(bolus)) for each day in the recent period is calculated. These daily sums are averaged (i.e., divided by n) to yield the TDM_{new}. At 406, the product of the new basal portion value (see 204 in FIG. 2) and TDM_{new} is calculated to get the adjusted daily basal delivery amount. This can be expressed as:

DBD_{new}=BR_{new}*TDM_{new} (Eq. 4)

At 408, the next basal delivery dosage is determined from the adjusted daily basal amount. Specifically, assume that the medicament delivery device 102 delivers basal medicament every cycle and that each cycle is five minutes long. Then, there are 180 cycles per day. That means that the adjusted basal delivery amount may be divided by 180 to determine the basal dosage per cycle (e.g., DBD_{new}/180).

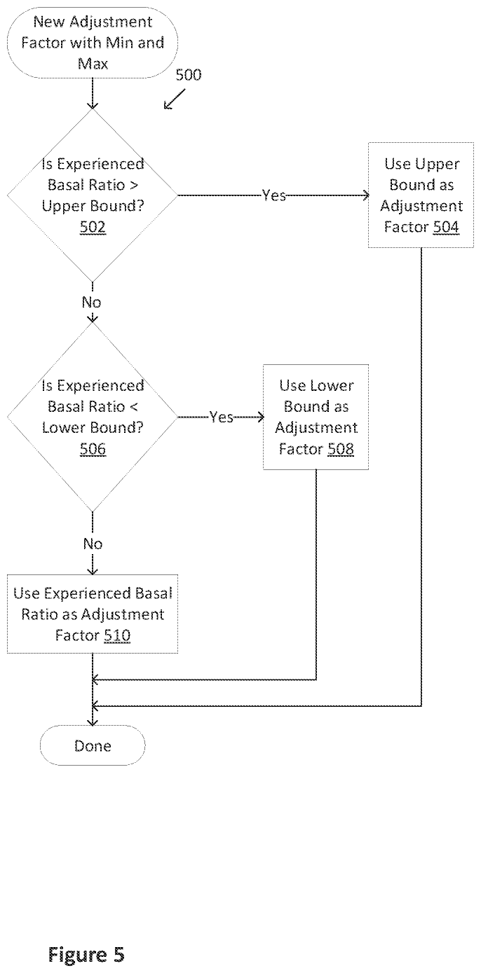

A number of different types of adjustment factors may be used in exemplary embodiments to adjust the basal portion value, such as described above. FIG. 5 depicts a flowchart 500 of illustrative steps that may be performed in exemplary embodiments where an adjustment factor is used that is bounded. An example of such an adjustment factor is

$$min ( max ( \frac{{M}_{basal}}{{TDM}_{new}} , \frac{1}{3} ) , \frac{2}{3} ) ,$$

where M_{basal} the average daily basal delivery amount for the time period. Thus, the adjustment factor is the smaller of ⅔ and the larger of the ratio of the average daily basal delivery amount to TDM_{new}, referred to hereafter as the “experienced basal ratio” and ⅓. So, the adjustment value has the value of the ratio but is bound by ⅓ to the low end and ⅔ to the high end. Hence if the ratio is less than ⅓, the adjustment factor assumes a value of ⅓, and if the ratio is greater than ⅔, the adjustment factor is ⅔. Plugging this adjustment factor into the new basal ratio factor equation of Equation 2, yields:

$${BR}_{new} = ( 1 - {A}_{r} * {N}_{days} ) * ( {BR}_{old} + {A}_{r} * {N}_{days} * min ( max ( \frac{{M}_{basal}}{{TDM}_{new}} , \frac{1}{3} ) , \frac{2}{3} ) . \qquad ( Eq . 5 )$$

The magnitude of the change to the basal ratio thus depends on the number of days in the time period and the difference in magnitude between the adjustment factor relative and BR_{old}.

As shown in the flowchart 500 of FIG. 5, at 502, a check is made whether the experienced basal ratio

$$( i . e . , \frac{{M}_{basal}}{{TDM}_{new}} )$$

is greater than the upper bound (e.g., ⅔). If so, at 504, the adjustment factor assumes the value of the upper bound. If not, at 506, a check is made whether the experienced basal ratio is less than the lower bound. If so, at 508, the adjustment factor is set as the lower bound (e.g., ⅓). Otherwise, at 510, the experienced basal ratio is the adjustment factor. It should be appreciated that in some exemplary embodiments only a single bound may be used, such as only an upper bound. In addition, values other ⅓ and ⅔ may be used for the bounds.

Another option for the adjustment factor is to have it be the average of the ideal value of the basal ratio of 0.5 and the experienced basal ratio. This formulation of adjustment factor may be expressed as

$$( \frac{\frac{{M}_{basal}}{{TDM}_{new}} + 0.5}{2} ) .$$

The equation for the new basal ratio may be expressed as:

$${BR}_{new} = ( 1 - {A}_{r} * {N}_{days} ) * {BR}_{old} + {A}_{r} * {N}_{days} * ( \frac{\frac{{M}_{basal}}{{TDM}_{new}} + 0.5}{2} ) . \qquad ( Eq . 6 )$$

This formulation of the adjustment factor moves the adjustment factor ½ way toward the ideal value of 0.5.

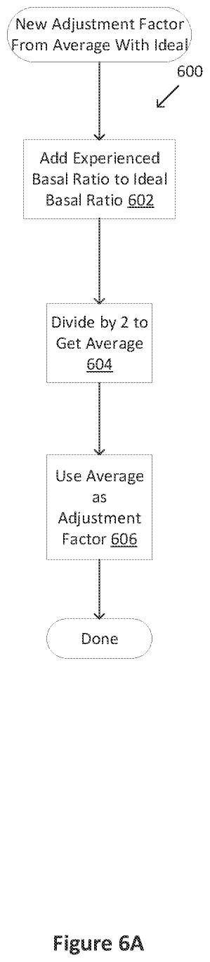

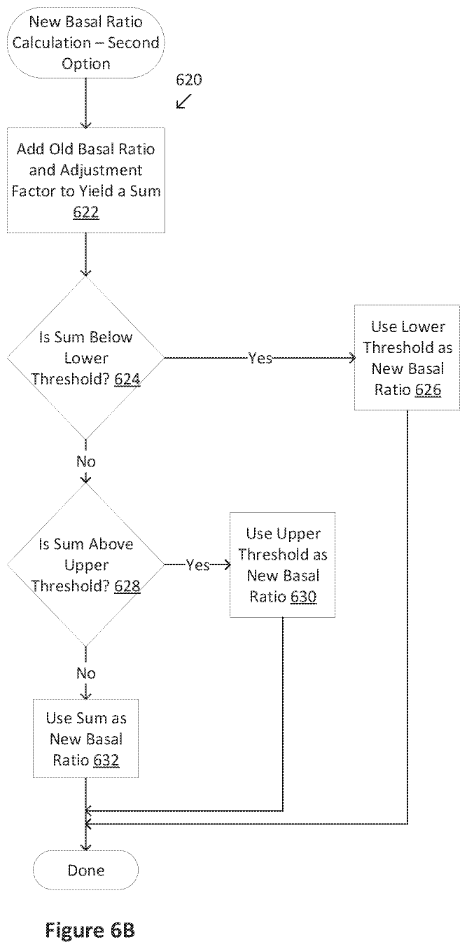

FIG. 6A depicts a flowchart 600 of illustrative steps that may be performed with this variety of adjustment factor. At 602, the experienced basal ratio is added to the ideal basal portion value

$$( e . g . , \frac{{M}_{basal}}{{TDM}_{new}} + 0.5 )$$

At 604, the sum is divided by two to yield an average. At 606, the average is used as the adjustment factor.

In addition, the adjustment factor may use fixed value options in some exemplary options. For example, the adjustment factor may assume a positive fixed value when the experienced basal ratio is above a threshold, a negative fixed value when the experienced basal ratio is below another threshold and have a zero or nominal value when between thresholds. One example formulation of this option is:

$$Adjustment Factor = \{ \begin{matrix}0.05 & \frac{{M}_{basal}}{{TDM}_{new}} > 0.5 \\ - 0.05 & \frac{{M}_{basal}}{{TDM}_{new}} \leq 0.5\end{matrix} . \qquad ( Eq . 7 )$$

In this instance, instead of the adjustment factor being weighted, it is just a constant + or − value. As such, the new basal ratio can be expressed as:

$${BR}_{new} = min ( max ( {BR}_{old} + Adjustment Factor , \frac{1}{3} ) , \frac{2}{3} ) . \qquad ( Eq . 8 )$$

FIG. 6B depicts a flowchart of illustrative steps that may be performed for calculating the new basal ratio in accordance with this second option as captured by Equation 8. Initially, at 622, the old basal ratio BR_{old} is added to the adjustment factor to yield a sum. At 624, a determination is made whether the sum is lower than the lower threshold. If so, at 626, the lower threshold is used as the new basal ratio. For example, if the lower threshold is ⅓ and the sum is lower than ⅓, the new basal ratio is set at ⅓. If the sum is greater than the lower threshold, then, at 628, a determination is made whether the sum is above the upper threshold. If so, at 630, the new basal ratio is set at the upper threshold (e.g., ⅔). If not, at 632, the sum is used as the new basal ratio.

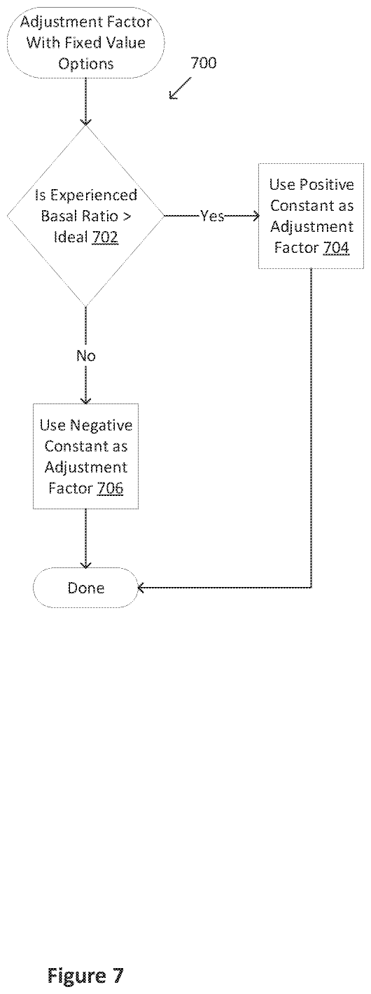

FIG. 7 provides a flowchart 700 of illustrative steps that may be performed in exemplary embodiments with such an option for the adjustment factor. At 702, a determination is made whether or not the experienced basal portion value (i.e., the experienced basal ratio) is greater than the ideal basal ratio (e.g., +0.05). If so, at 704, the positive adjustment factor (e.g., 0.05) is used. If not, then the negative adjustment factor (e.g., −0.05) is used.

An additional option is to use an adjustment value with a scaling value. The scaling value may be 0.5, for example. An illustrative expression for the adjustment factor with this option is:

$$Adjustment Factor = \{ \begin{matrix}0.5 + \sqrt{\frac{{M}_{basal}}{{TDM}_{new}} - 0.5} & \frac{{M}_{basal}}{{TDM}_{new}} > 0.5 \\ 0.5 - \sqrt{0.5 - \frac{{M}_{basal}}{{TDM}_{new}}} & \frac{{M}_{basal}}{{TDM}_{new}} \leq 0.5\end{matrix} . \qquad ( Eq . 9 )$$

This illustrative adjustment factor scales the adjustment factor relative to the ideal value of 0.5 based on the square root of the difference between the experienced basal ratio and the ideal value. Equation 2 may be applied to determine the new basal ratio.

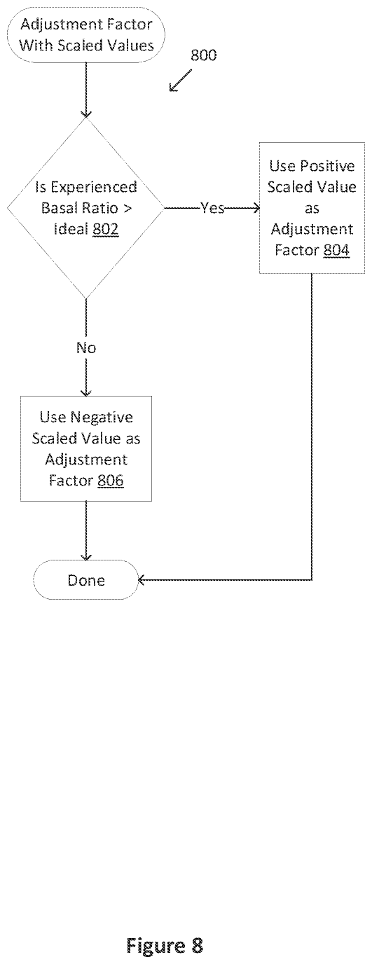

FIG. 8 depicts a flowchart 800 of illustrative steps that may be performed when this option for determining the adjustment factor is used. At 802, a determination is made whether the experienced basal ratio

$$( e . g . , \frac{{M}_{basal}}{{TDM}_{new}} )$$

is greater than the ideal basal ratio of 0.5. If so, at 804, the positive scaled value

$$( e . g . , 0.5 + \sqrt{\frac{{M}_{basal}}{{TDM}_{new}} - 0.5} )$$

is used as the adjustment factor. If not, at 806, the negative scaled value

$$( e . g . , 0.5 - \sqrt{0.5 - \frac{{M}_{b a s a l}}{T D {M}_{n e w}}} )$$

is used as the adjustment factor.

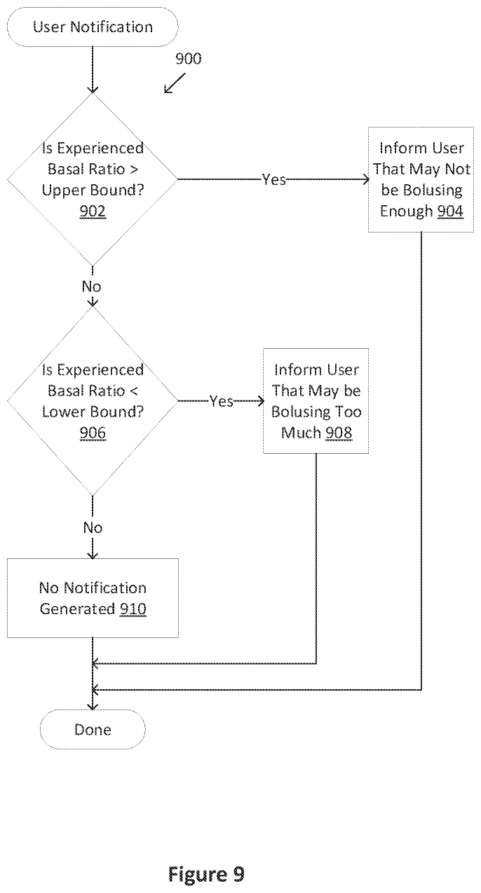

The exemplary embodiments may provide notifications to the user relating to the experienced basal ratio being outside a desirable range. FIG. 9 depicts a flowchart of illustrative steps that may be performed in exemplary embodiments in providing notifications. At 902, a determination is made whether the experienced basal ratio is above an upper bound. If so, at 904 a notification is sent to the user that the user may not be bolusing enough. The notification may be displayed on display 109. The notification may also be sent via messaging mechanisms like text messaging or email. Next, at 906, a determination may be made whether the experienced basal ratio is below the lower bound. If so, at 908, a notification may be sent to the user that the user may be bolusing too much. If not, at 910, no notification may be generated.

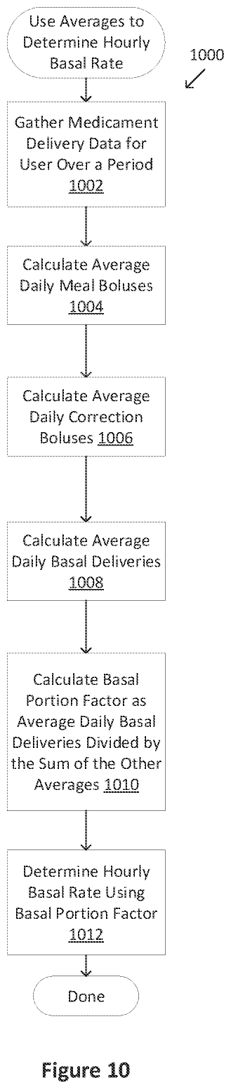

Another approach to adapting the basal delivery rate to the customized needs of the user is depicted in FIG. 10. FIG. 10 depicts a flowchart of illustrative steps that may be performed in exemplary embodiments to determine an hourly basal rate for a user based on historical averages over a period. At 1002, medicament delivery data for a user is gathered over a period. The medicament delivery data may include that for basal medicament deliveries, meal bolus medicament deliveries that are delivered to address the increases in blood glucose due to ingestion of carbohydrates and correction bolus medicament deliveries to address high blood glucose.

At 1004, the average daily meal boluses for the user over the period is calculated. If there are m days in the period, this average may be calculated as:

$$Average M ( meal ) = \frac{1}{m} {\sum }_{k = 0}^{m} {Medicament}_{k} ( meal ) \qquad ( Eq . 10 )$$

where Medicamentₖ(meal) is the amount of meal bolus medicament that was delivered to the user on day k and Average M(meal) is the average meal bolus medicament amount that was delivered per day over the period of m days.

At 1006, the average daily correction bolus amount for the user over the period is calculated. A suitable equation for this average is:

$$Average M ( correction ) = \frac{1}{m} {\sum }_{k = 0}^{m} {Medicament}_{k} ( correction ) \qquad ( Eq . 11 )$$

where Medicamentₖ(correction) is the amount of correction bolus medicament that was delivered to the user on day k and Average M(correction) is the average correction bolus medicament amount that was delivered per day over the period of m days.

At 1008, the average daily basal medicament delivery amount is calculated. The average may be calculated as:

$$Average M ( basal ) = \frac{1}{m} {\sum }_{k = 0}^{m} {Medicament}_{k} ( basal ) \qquad ( Eq . 12 )$$

where Medicamentₖ(basal) is the amount of medicament that was delivered as basal deliveries to the user on day k and Average M(basal) is the average basal medicament amount that was delivered per day over the period of m days.

At 1010, the basal portion factor may be calculated as the average daily basal deliveries divided by the sum of the other averages calculated at 1004, 1006 and 1008. The basal portion factor relates to the new basal ratio adapted to data gathered over the period. This calculation may be expressed as:

$$basal por tion facto r = \frac{Average M ( basal )}{Average M ( basal ) + Average M ( meal ) + Average M ( correction )} . \qquad ( Eq . 13 )$$

At 1012, the hourly basal rate is determined using the basal portion factor. The hourly basal rate may be expressed as:

$$\frac{B a s a l}{hour} = \frac{1}{24} * TDI * basal portion factor . \qquad ( Eq . 14 )$$

The exemplary embodiments may also adjust the bolus amounts delivered to the user based on the basal portion value. Given the wide range of possible deviations in the user's daily bolus needs, rather than directly translating the user's immediate basal proportions of insulin delivery into changing the user's basal proportions, the exemplary embodiments may increment the basal proportions by a fixed amount if the user's insulin needs vary away from the norm (50/50 split) by more than 10% or more (0.05 is 10% of 0.5) towards one direction. The adjustment in the basal portion value may be expressed, for example, as:

$${b}_{factor} = 1 + \{ \begin{matrix}- 0.05 & basal por t i on value \leq 0.45 \\ 0 & 0.4 5 < basal p o r t i on value < 0.55 \\ 0.05 & 0.5 5 \leq basal p o rtion value\end{matrix} \begin{matrix} \\ . \\ \end{matrix} \qquad ( Eq . 15 )$$

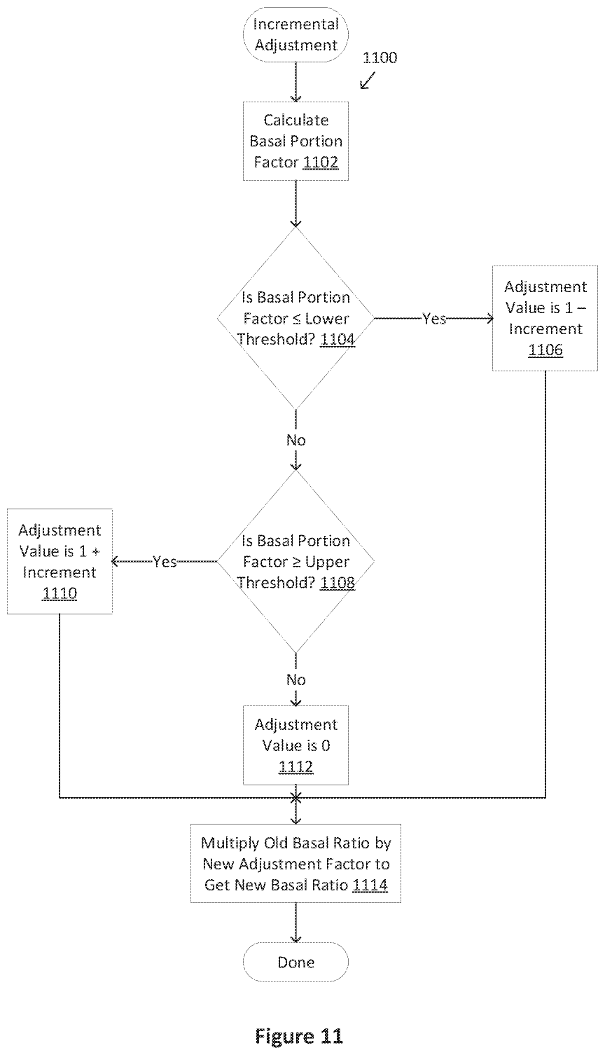

Hence, as shown in the flowchart 1100 of FIG. 11, at 1102, the basal portion factor may be calculated, such as by using Equation 13. A determination is made at 1104 of whether the basal portion factor is less than or equal to the lower threshold (e.g., 0.45). Such a value would indicate that the actual basal portion factor for the user is less than 0.45 and should be adjusted downward. If so, at 1106, the adjustment value is 1−increment (e.g., 0.05). If not, at 1108, a check is made whether the basal portion factor is greater than or equal to the upper threshold (e.g., 0.55) and should be adjusted upward. If so, at 1110, the adjustment value is adjusted to be 1+increment. If not, at 1112, the basal portion factor is close to ideal, and the adjustment value is set at 0. At 1114, the old basal ratio b_{old} is multiplied by the new adjustment factor b_{factor} to get the new basal ratio b_{new}:

b_{new}=b_{old}·b_{factor} (Eq. 16).

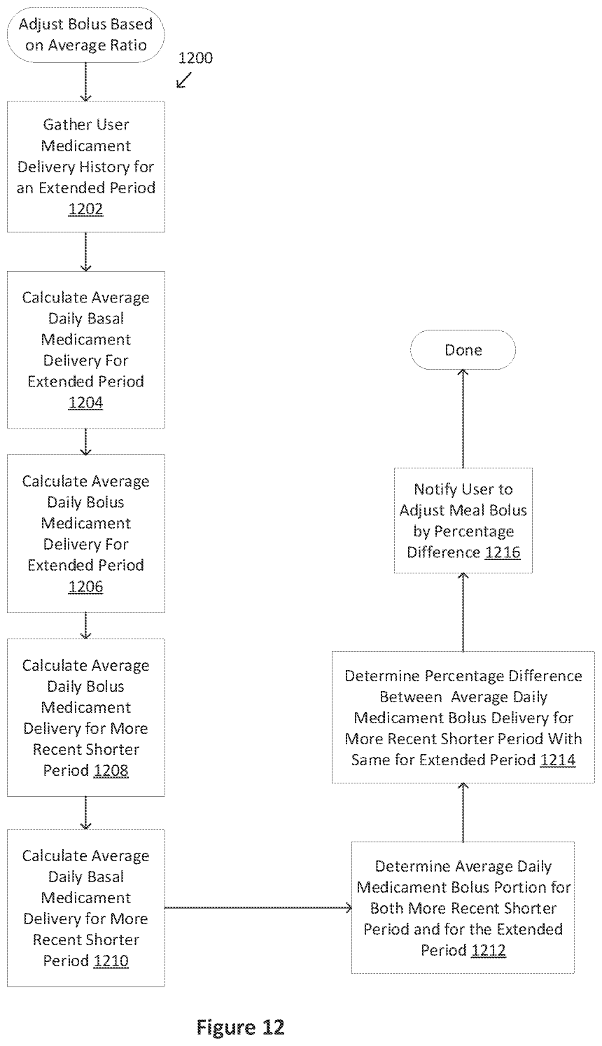

Another exemplary embodiment uses deviations from a standard basal correction factor to detect over-bolus or under-bolus from historical data. In scenarios where a dynamically calculated basal correction factor is not available, the under bolus and over bolus amounts can be determined, and the user can be warned. FIG. 12 depicts a flowchart 1200 of illustrative steps that may be performed in such an exemplary embodiment. At 1202, user medicament delivery history is gathered for an extended period and a more recent shorter period of time. This data may be used to establish averages, and the averages may be used to determine over-bolusing and under-bolusing. At 1204, the average daily basal medicament delivery for the user over the extended period is calculated, such as by using Equation 12. At 1206. The average daily bolus medicament amount for the user for the extended period is calculated, such as by summing Equations 10 and 11. At 1208, the average daily bolus medicament delivery to the user for the shorter more recent period of time is calculated. At 1210, the average daily basal medicament delivery to the user for the shorter period of time is calculated. At 1212, the average medicament bolus portion for the shorter period and for the extended period are calculated. These are the ratio of average daily bolus to TDM for the respective shorter and extended periods. At 1214, the percentage difference between the average medicament bolus portion for the use for the shorter period and the average medicament bolus portion for the use for the extended period is determined. At 1216, the user is sent a notification, such as via display 109 or 127, to adjust the bolus amount by the determined percentage difference. The percentage difference may be a positive or negative value. For example, suppose that TDM is 140, and the bolus portion for the extended period is 100/140 and 105/140 for the shorter period. The percentage difference is roughly +3.5%.

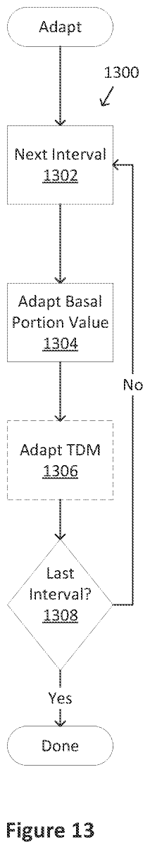

As was described above, the basal portion value and/or TDM may be adapted over shorter time intervals than discussed above, such as over hourly time intervals. FIG. 13 depicts a flowchart 1300 of illustrative steps that may be performed in exemplary embodiments to adapt the basal portion value and TDM for a user. At 1302, a next interval is reached. The interval may be, for example, a one hour interval, a three hour interval, a six hour interval or a twelve hour interval. Intervals may be shorter, such as a half hour in length. The arrival of the next interval is a trigger to adapt the basal portion value at 1304. The TDM may optionally be adapted as well at 1306. A check may be made at 1308 to see if the last interval has been reached. For example, the interval may be the last interval if the interval is the last one before the medicament delivery device is replaced. If not, the process repeats with the arrival of the next interval at 1302.

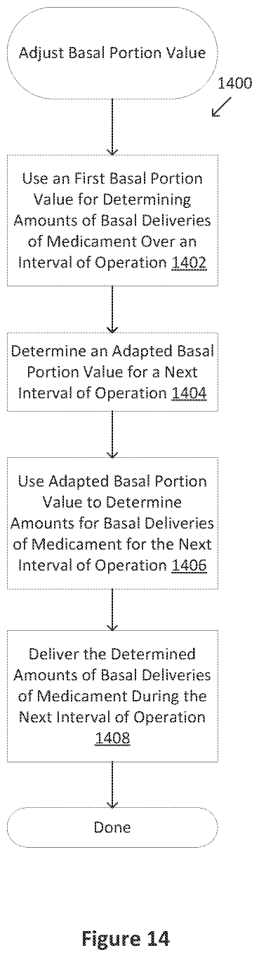

FIG. 14 depicts a flowchart 1400 of illustrative steps that may be performed in exemplary embodiments to adjust the basal portion value for the shortened time intervals. At 1402, a first basal portion value is used for determining amounts of basal deliveries of medicament over an interval of operation. At 1404, for the next interval of operation, an adapted basal portion value is determined. The adapted basal portion value may reflect changes in medicament sensitivity of the user as captured by recent medicament deliveries to the user. For example, where the medicament being delivered is insulin, factors like exercise, menstrual cycle, time of day and the like, may affect insulin sensitivity. At 1406, the adapted basal portion value is used to determine the amount of medicament delivered to the user for the next interval of operation. At 1408, the control application 116 or 120 causes the determined basal medicament amounts to be delivered to the user over the next interval of time.

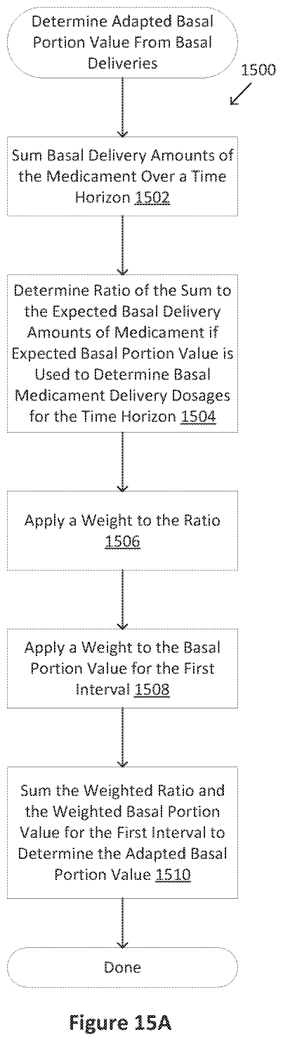

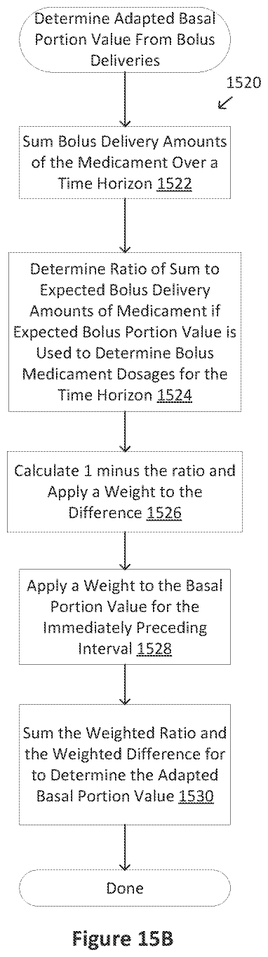

The adapted basal portion value may be determined (see 1406) based on recent medicament delivery history of the user. FIG. 15A depicts a flowchart 1500 of illustrative steps that may be performed in exemplary embodiments to determine the adapted basal portion value from recent medicament delivery history of the user. The following equation may be used to determine the adapted basal portion value for the user

$${S}_{i , b} ( k ) = 0.95 \cdot {S}_{i} ( k - 1 ) + 0.05 \frac{{\sum }_{i = 1}^{N} {I}_{B a s a l} ( i )}{{S}_{i} ( 1 ) \cdot ( \frac{T D M}{\frac{288}{N}} )} \qquad ( Eq . 16 )$$

where k is a time interval index, Sᵢ(k−1) is the basal portion value for the immediately previous time horizon, Sᵢ(1) is the initial expected basal portion value (e.g., 0.5), N is the number of cycles in the time horizon, and I_{Basal}(i) is the amount of basal medicament delivered at cycle i of the time horizon.

Thus, at 1502, the basal delivery amounts of medicament over the time horizon N are summed (i.e. Σᵢ₌₁^{N} I_{Basal}(i)). At 1504, the ratio of the sum to the expected basal delivery amounts if the expected basal portion value (i.e., Sᵢ(1)) is used to determine the basal medicament delivery dosages for the time horizon

$$( i . e . , {S}_{i} ( 1 ) \cdot ( \frac{T D M}{\frac{288}{N}} ) ) \qquad ( 1 )$$

is determined. This ratio represents an indication of how much the basal insulin delivery history for the time horizon differs from the expected basal delivery dosages for the time horizon. The difference can be reflective of changing insulin sensitivity of the user. At 1506, a weight (e.g., 0.05 in Equation 16) is applied to the ratio. At 1508, a weight (e.g., 0.95 in Equation 16) is applied to the basal portion value for the first interval (e.g., Sᵢ(1)). The weights dictate how quickly the basal portion value adapts to the recent basal delivery history and other weights may be used. With the illustrative weights in Equation 16, the recent basal delivery history figures in only five percent of the adapted basal portion value each time an adaptation of the basal portion value occurs. At 1510, the weighted ratio and the weighted basal portion value for the first interval to determine the adapted basal portion value.

The recent bolus delivery history may be used instead of the recent basal delivery history to adapt the basal portion value. There is a relationship between the basal portion value and the bolus portion value, which represents the portion of TDM attributable to manual bolus deliveries. In particular, the basal portion value equals 1 minus the bolus portion value. This relationship may be used to determine the basal portion value. A suitable equation for determining the adapted basal portion value for a user from recent bolus delivery history is:

$${S}_{i , m} ( k ) = 0.95 \cdot {S}_{i} ( k - 1 ) + 0.05 ( 1 - \frac{{\sum }_{i = 1}^{N} {I}_{manual} ( i )}{( 1 - {S}_{i} ( 1 ) ) \cdot \frac{TDI}{288 / N}} ) , \qquad ( Eq . 17 )$$

where Iₘₐₙᵤₐₗ(i) is the amount of manual insulin delivered in cycle i.

FIG. 15B depicts a flowchart 1520 of illustrative steps that may be performed in exemplary embodiments to adapt the basal portion value for a user. At 1522, the bolus delivery amounts that were manually delivered to the user over the time horizon are summed for all of the cycles of the time horizon (i.e., Σᵢ₌₁^{N} Iₘₐₙᵤₐₗ(i)). The sum constitutes the total amount of medicament delivered in manual boluses to the user over the time horizon. At 1524, a ratio of the sum with the expected bolus delivery amounts of medicament if the expected bolus portion value is used to determine bolus dosages for the time horizon

$$( i . e . , ( 1 - {S}_{i} ( 1 ) ) \cdot \frac{TDI}{288 / N} ) .$$

At 1526, 1 minus the ratio is calculated to determine the recent amount of basal medicament deliveries over the time horizon relative to the expected amount of basal medicament deliveries over the time horizon, and a weight (e.g., 0.05) is applied to the calculated difference. At 1528, a weight (e.g., 0.95) is applied to the basal portion value for the immediately preceding interval (i.e., Sᵢ(k−1)). At 1530, the weighted ratio and the weighted difference ae summed to determine the adapted basal portion value (i.e., S_{i,m}(k)) for the time interval.

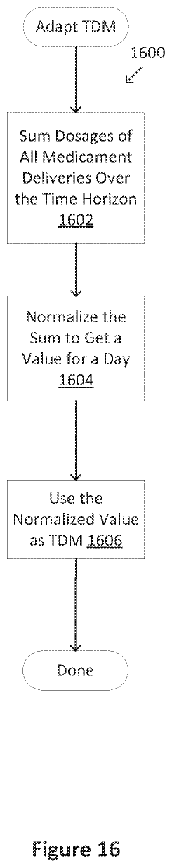

The TDM value may also be adapted frequently, even for each cycle or for each interval. FIG. 16 depicts a flowchart 1600 of steps that may be performed in exemplary embodiments to adapt TDM for a user. The dosages of all medicament deliveries to a user (i.e., all manual deliveries and all automated deliveries) over a recent time horizon are summed at 1602. In some embodiments, the recent time horizon should be over one day long. Thus, if cycles for the medicament delivery device are 5 minutes long, then the time horizon should span more than 288 cycles (i.e., the number of cycles in a day). At 1604, the resulting sum is normalized to get a value for a day. If the time horizon M is greater than 288 cycles, then the sum can be divided by M/288 to normalize the sum. At 1606, the normalized sum is used as the TDM.

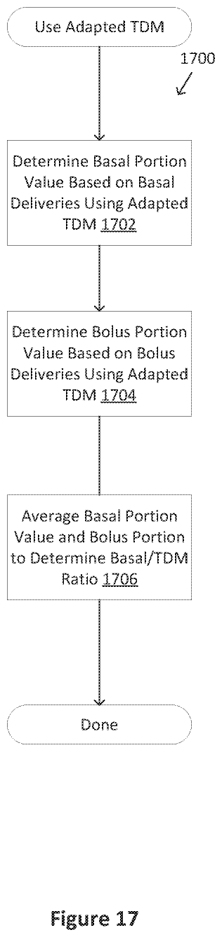

The adapted TDM (or TDI in the case of insulin) may be used in determining the adapted basal portion of TDM. FIG. 17 depicts a flowchart 1700 of illustrative steps that may be performed in exemplary embodiments to use the adapted TDM in determining the basal portion value. At 1702, the basal portion value is determined using recent basal deliveries with an adapted TDM value. Substituting the formulation detailed above into Equation 16 for determining the basal portion value from recent basal deliveries yields the following equation:

$${S}_{i , b} ( k ) = 0.95 \cdot {S}_{i} ( k - 1 ) + 0.05 \frac{{\sum }_{i = 1}^{N} {I}_{B a s a l} ( i )}{{S}_{i} ( 1 ) \cdot \frac{( {\sum }_{i = 1}^{{M}_{> 288}} {I}_{B a s a l} ( i ) + {I}_{m a n u a l} ( i ) ) / ( \frac{M}{288} )}{288 / N}} . \qquad ( Eq . 18 )$$

At 1704, the basal portion value is determined using recent bolus deliveries with an adapted TDM value. Substituting the formulation detailed above into Equation 17 for determining the basal portion value from recent bolus deliveries yields the following equation:

$${S}_{i , m} ( k ) = 0.95 \cdot {S}_{i} ( k - 1 ) + 0.05 ( 1 - \frac{{\sum }_{i = 1}^{N} {I}_{manual} ( i )}{( 1 - {S}_{i} ( 1 ) ) \cdot \frac{( {\sum }_{i = 1}^{{M}_{> 288}} {I}_{B a s a l} ( i ) + {I}_{m a n u a l} ( i ) ) / ( \frac{M}{288} )}{288 / N}} ) \qquad ( Eq . 19 )$$

The operative basal portion may be determined at 1706 by averaging the basal portion values of 1704 and 1706. This may be expressed as

$${B}_{i} ( k ) = \frac{{S}_{i , b} ( k ) + {S}_{i , m} ( k )}{2} \cdot TDM . \qquad ( Eq . 20 )$$

Alternatively, with the adapted TDM, the operative basal portion may be expressed as

$${B}_{i} ( k ) = \frac{{S}_{i , b} ( k ) + {S}_{i , m} ( k )}{2} \cdot ( {\sum }_{i = 1}^{{M}_{> 288}} {I}_{B a s a l} ( i ) + {I}_{m a n u a l} ( i ) ) / ( \frac{M}{288} ) . \qquad ( Eq . 21 )$$

While exemplary embodiments have been described herein, it should be appreciated that various changes in form and detail may be made without departing from the intended scope as defined in the appended claims.

1. A medicament delivery system, comprising:
a non-transitory storage for storing computer programming instructions; and
a processor for executing the computer programming instructions, said computer programming instructions causing the processor to:
calculate a new basal ratio for a user that specifies what portion of total daily medicament for the user is to be delivered by basal medicament deliveries based on:
a previous basal ratio for the user,
an adjustment, comprising:
a ratio of amounts of a medicament delivered as basal medicament deliveries to the user via an automated medicament delivery device over a period to a total amount of medicament delivered to the user over the period, and
at least one adjustment restriction that restricts how much the new basal ratio may vary from the previous basal ratio; and
determine a dosage for a next basal medicament delivery by the automated medicament delivery device to the user using the new basal ratio.

2. The medicament delivery system of claim 1, wherein the medicament is insulin.

3. The medicament delivery system of claim 2, wherein the determining the dosage for the next basal medicament delivery comprises multiplying a total daily insulin for the user by the new basal ratio.

4. The medicament delivery system of claim 1, wherein at least one adjustment restriction includes a maximum and/or minimum value that the adjustment may assume.

5. The medicament delivery system of claim 1, wherein the at least one adjustment restriction limits a difference between the previous basal ratio and the new basal ratio to a predetermined fixed amount.

6. The medicament delivery system of claim 1, wherein the at least one adjustment restriction is a scaling factor that scales the adjustment.

7. The medicament delivery system of claim 1, wherein the medicament includes at least one of a glucagon like peptide-1 (GLP-1) agonist, pramlintide or another agent that affects a glucose level of the user.

8. A medicament delivery system, comprising:
a non-transitory storage for storing computer programming instructions; and
a processor for executing the computer programming instructions, said computer programming instructions causing the processor to:
determine an average amount of insulin delivered to a user per day to compensate for carbohydrates ingested in meals;
determine an average amount of insulin delivered to the user per day to correct for high glucose experienced by the user;
determine an average amount of insulin delivered by the user as basal deliveries;
compute the sum of the average amount of insulin delivered to a user per day to compensate for carbohydrates ingested in meals, the average amount of insulin delivered to the user per day to correct for high glucose experienced by the user and the average amount of insulin delivered by the user as basal deliveries;
determine a basal portion factor as a ratio of the average amount of insulin delivered by the user as basal deliveries to the sum; and
determine an updated basal delivery rate for the user using the basal portion factor.

9. The medicament delivery system of claim 8, the determining of the updated basal delivery rate for the user comprises determining an updated hourly basal delivery rate for the user.

10. The medicament delivery system of claim 9, wherein the determining the hourly basal delivery rate comprises determining a product of total daily insulin (TDI) for the user with the basal portion factor and dividing the product by 24 to get the hourly basal delivery rate.

11. The medicament delivery system of claim 8, further comprising updating the basal portion factor and updating the basal delivery rate for the user using the updated basal portion factor.

12. The medicament delivery system of claim 11, wherein the updating the basal portion factor comprises increasing the basal portion factor by a first predetermined fixed amount if the basal portion factor is below a threshold.

13. The medicament delivery system of claim 11, wherein the updating the basal portion factor comprises decreasing a basal portion factor by a second predetermined fixed amount if the basal portion factor is above a threshold.

14. A medicament delivery system, comprising:
a display;
a non-transitory storage for storing computer programming instructions; and
a processor for executing the computer programming instructions, said computer programming instructions causing the processor to:
compare an average meal bolus portion of total daily insulin for a patient over most recent days with a historical average meal bolus portion of total daily insulin for the patient;
if the average meal bolus portion of total daily insulin over most recent days is higher than the historical average meal bolus portion of total daily insulin by a first percentage, generating a suggestion on the display to the user to lower an amount of insulin for meal boluses by the first percentage; and
if the average meal bolus portion of total daily insulin over most recent days is lower than the historical average meal bolus portion of total daily insulin by a second percentage, generating a suggestion on the display to the user to increase an amount of insulin for meal boluses by the first percentage.

15. The medicament delivery system of claim 14, wherein the medicament delivery system includes one of an insulin pump, a controller for an insulin pump or a portable computing device.

16. The medicament delivery system of claim 14, wherein the computer programming instructions further cause the processor to calculate a historical average basal daily insulin delivery to the patient.

17. The medicament delivery system of claim 16, wherein the computer programming instructions further cause the processor to calculate a historical average meal bolus daily insulin delivery to the patient.

18. The medicament delivery system of claim 17, wherein the computer programming instructions further cause the processor to calculate historical average total daily insulin for the patient as the sum of the historical average basal daily insulin delivery to the patient and the historical average meal bolus daily insulin delivery to the patient.

19. The medicament delivery system of claim 17, wherein the computer programming instructions further cause the processor to calculate the historical average meal bolus portion of total daily insulin for the patient as a ratio of historical average meal bolus daily insulin delivery to the patient to the historical average total daily insulin for the patient.

20. The medicament delivery system of claim 17, wherein the first percentage equals the second percentage.
